(19) United States
(12) Patent Application Publication
(10) Pub. No.: US 2023/0338936 A1
(43) Pub. Date: Oct. 26, 2023

(54) STANNOUS SALT COMPLEX CATALYST AND METHOD FOR PRODUCING L-LACTIDE BY USING THE SAME

(71) Applicant: JIANGSU JINGHONG NEW MATERIALS TECHNOLOGY CO., LTD, Jiangsu (CN)

(72) Inventors: Di WU, Suqian, Jiangsu (CN); Peilong WU, Suqian, Jiangsu (CN); Yinqiu LU, Suqian, Jiangsu (CN); Jianguo CHEN, Suqian, Jiangsu (CN); Xinwei LIU, Suqian, Jiangsu (CN); Ge SONG, Suqian, Jiangsu (CN); Changwang CAI, Suqian, Jiangsu (CN)

(73) Assignee: JIANGSU JINGHONG NEW MATERIALS TECHNOLOGY CO., LTD, Jiangsu (CN)

(21) Appl. No.: 17/636,297

(22) PCT Filed: Aug. 18, 2021
(86) PCT No.: PCT/CN2021/113174

§ 371 Date: Feb. 17, 2022

(30) Foreign Application Priority Data
Dec. 15, 2020 (CN) 202011480932.2

(51) Int. Cl. B01J 31/18; C07C 67/08; B01J 35/12; B01J 37/04; B01J 31/04

(52) U.S. Cl. CPC B01J 31/185; B01J 31/04; B01J 35/12; B01J 37/04; C07C 67/08; B01J 2231/14; B01J 2531/42

(57) ABSTRACT

The present disclosure provides a stannous salt complex catalyst and a method for producing L-lactide by using the same. The catalyst is obtained by reacting stannous salt and phosphite in a solvent, which can effectively inhibit the oxidation of divalent stannous ions to tetravalent tin ions, effectively reduce the conversion rate of meso-lactide, increase the yield of L-lactide, and obtain nearly white L-lactide. In the crude lactide produced after the depolymerization reaction, the content of L-lactide is more than or equal to 95 %; the content of meso-lactide is less than or equal to 3.8 %, while ensuring that the color of the residual liquid is close to milky white.

### CROSS REFERENCE TO RELATED APPLICATION

This patent application claims the benefit and priority of Chinese Patent Application No. 202011480932.2 filed on Dec. 15, 2020, the disclosure of which is incorporated by reference herein in its entirety as part of the present application.

### TECHNICAL FIELD

The present disclosure belongs to the technical field of L-lactide preparation, and specifically relates to a stannous salt complex catalyst and a method for producing L-lactide by using the same.

### BACKGROUND ART

At present, the commonly used cracking catalysts for preparing lactide are mainly metals such as zinc, tin and their metal salts, among which the most widely used are stannous salts, including stannous octoate, stannous chloride, and stannous sulfate. However, stannous salt is very easy to be oxidized, especially under the conditions of cracking reaction (>200° C.), which will be rapidly oxidized and decomposed to generate tetravalent tin salt, thereby losing catalytic activity, and tin salt will also cause the racemization of lactide, which increases the amount of meso-lactide. At the same time, it will cause the color of lactide and residual liquid to deepen.

CN101903370B discloses a stannous phosphite as a cracking catalyst for synthesizing lactide. The reducibility of phosphorous acid prevents the oxidation of stannous ions into tin ions, thereby reducing the conversion rate of meso-lactide and improving the hue of lactide. However, phosphite is more toxic and can generate highly toxic phosphine under acidic conditions, which is less safe. CN110156745A discloses a composite catalyst of zinc compound and / or tin compound and alkali metal compound, but the addition of alkali metal compound cannot improve the oxidation of stannous salt to tin salt. GB2331986A discloses the compound use of stannous octoate and phosphite stabilizer for preparing lactide. However, phosphite, as a stabilizer, does not coordinate with stannous salt, which could not completely prevent stannous salt from being oxidized to stannous salt, only improves the hue of lactide, but does not reduce the conversion rate of meso-lactide.

### SUMMARY

The purpose of the present disclosure is to provide a method for preparing a stannous salt complex catalyst and a method for producing lactide using the same. The preparation process of the catalyst provided by the present disclosure is simple and pollution-free, has low requirements on equipment, and is convenient for industrialization. The catalyst has high stability at high temperature, stannous salt will not be oxidized into tetravalent tin salt, and the L-lactide produced by using the catalyst provided by the present disclosure has a good hue, a small content of meso-lactide and a good hue of the residual liquid.

In order to achieve the above purpose, the present disclosure adopts the following technical schemes:
A stannous salt complex catalyst, wherein comprising the following raw materials:
   (1) A stannous salt
   (2) A phosphite
   (3) A solvent
Wherein, the mass ratio of stannous salt to phosphite is 5: 1-20: 1, the mass ratio of stannous salt to solvent is 2: 1-1: 5, and the preparation steps of the stannous salt complex catalyst are as follows:
   (1) Dissolving the stannous salt in the solvent and heating reflux for 30 min to fully dissolve the stannous salt to obtain a stannous salt solution;
   (2) Under the protection of nitrogen, adding the phosphite dropwise to the stannous salt solution, and performing a reflux reaction for 2 h;
   (3) After the reaction, extracting the solvent from the system and recycling, and obtaining a final light yellow viscous liquid which is the stannous salt complex catalyst.

In some embodiments, the stannous salt is one or more selected from the group consisting of stannous isooctanoate, stannous chloride, stannous sulfate, stannous oxalate and stannous phosphate.

In some embodiments, the phosphite is one or more selected from the group consisting of triphenyl phosphite, tridecyl phosphite, diphenyl phosphite, and diisodecyl pentaerythritol diphosphite.

In some embodiments, the solvent is one or two or more of benzene, toluene, ethyl acetate, acetone and xylene.

In some embodiments, the mass ratio of stannous salt to phosphite is 5: 1, and the mass ratio of stannous salt to solvent is 2: 1.

The present disclosure provides a method for preparing the stannous salt complex catalyst described in above technical schemes, wherein comprising the following steps:
(1) Dissolving the stannous salt in the solvent and heating reflux for 30 min to fully dissolve the stannous salt to obtain a stannous salt solution;
(2) Under the protection of nitrogen, adding the phosphite dropwise to the stannous salt solution, and performing a reflux reaction for 2 h;
(3) After the reaction, extracting the solvent from the system and recycling, and obtaining a final light yellow viscous liquid which is the stannous salt complex catalyst.

The present disclosure provides a method for producing L-lactide using the stannous salt complex catalyst, wherein comprising the following steps:
(1) Continuously adding a L-lactic acid aqueous solution with a mass concentration of 80-98% to a first reactor with a rectification device, removing the free water and bound water at a temperature of 80-120° C. and a vacuum of -0.05--0.09 MPa, separating the water and lactic acid vapor produced in the reaction by a rectification tower, introducing the water vapor at the top of the tower into a collection tank after being condensed, controlling the degree of polymerization of oligolactic acid to 2-8, and continuously transporting the oligolactic acid to a second reactor through a pump;
(2) Equipping the second reactor with a rectification device, subjecting the oligomeric lactic acid to a further polymerization at a temperature of 120-170° C. and a vacuum degree of -0.1 MPa, controlling the degree of polymerization of the oligomeric lactic acid to 8-25, continuously transporting the oligomeric lactic acid and the continuously injected stannous salt complex catalyst to a static mixer through a pump and mixing uniformly, and then transporting a resulting mixture to a third reactor;
(3) Subjecting the oligomeric lactic acid to a cracking reaction in the third reactor with a reaction temperature of 200-230° C. and a pressure of 50-500 Pa, condensing and collecting a crude lactide vapor generated by the cracking of oligomeric lactic acid in a crude lactide storage tank, introducing an unevaporated residual liquid into a residual liquid collection tank to purify the crude lactide to obtain L-lactide with a purity required for melt polymerization.

In some embodiments, the addition amount of the stannous salt complex catalyst is 0.5-5 % of the mass of the oligomeric lactic acid with a degree of polymerization of 8-25.

In some embodiments, the third reactor is a cage evaporation reactor, a scraper type film evaporator, a falling film evaporator or a rising film evaporator.

In some embodiments, the crude lactide is purified by one or several methods of melt crystallization, rectification and solvent recrystallization.

The beneficial effects of the present disclosure are as follows: the present disclosure uses the coordination reaction of stannous salt and phosphite to prepare a catalyst, which can significantly improve the stability of the catalyst at high temperature, and can effectively inhibit the oxidation of divalent stannous ions to tetravalent tin ions; it can effectively reduce the conversion rate of meso-lactide, increase the yield of L-lactide, and obtain nearly white L-lactide. In the crude lactide produced after the depolymerization reaction, the content of L-lactide is more than or equal to 95 %; the content of meso-lactide is less than or equal to 3.8 %, while ensuring that the color of the residual liquid is close to milky white.

### DETAILED DESCRIPTION OF THE EMBODIMENTS

The present disclosure will be further described below in conjunction with specific embodiments, but the present disclosure is not limited to these embodiments only.

### Example

### 1. Preparation of Stannous Salt Complex Catalyst:

100 g of stannous isooctanoate was added to 50 g of toluene, dissolved under reflux at 110° C. for 30 min, 20 g triedecyl phosphite was added to the mixed solution of stannous isooctanoate and toluene, and the reflux reaction was continued for 2 h. After the reaction, toluene in the reaction system was extracted under vacuum and recycled. The resulting light yellow viscous liquid was the stannous salt complex catalyst.

### 2. Preparation of Oligomeric Lactic Acid:

A L-lactic acid aqueous solution with a mass concentration of 90 % was added to the first reactor at a rate of 15 kg/h. The first reactor was equipped with a rectifying tower. The reaction temperature was kept at 120° C., the vacuum degree was -0.08 MPa, the time was 2 h, the degree of polymerization of the oligomeric lactic acid was controlled to 6-8, and the reaction product was continuously transported to the second reactor by a pump.

The second reactor was also equipped with a rectification device. The oligomeric lactic acid was further polymerized at a temperature of 160° C. and a vacuum degree of -0.1 MPa. The degree of polymerization of the oligomeric lactic acid was controlled to 15-20. The reaction product was continuously transported to the third reactor by a pump.

### 3. Cracking of the Oligomeric Lactic Acid to Produce Crude Lactide:

The oligomeric lactic acid from the second reactor and the continuously injected stannous salt complex catalyst were mixed in a static mixer, and then added to the rising film evaporator with an evaporation area of 0.8 m² at a rate of 13 kg/h, wherein the amount of stannous salt complex catalyst used was 1% of the mass of oligomeric lactic acid, the reaction temperature of the evaporator was 230° C., and the pressure was 500 Pa. The crude lactide was continuously generated, and condensed and collected in the lactide storage tank. The unevaporated heavy components were introduced into the residual liquid collection tank. The components of the crude lactide are shown in Table 1.

### 4. Recycling of Residual Liquid:

The residual liquid produced by cracking was mixed with oligomeric lactic acid and stannous salt complex catalyst in a static mixer by continuous injection, and then re-added to the rising film evaporator. After a month of circulating operation, the components of crude lactide are shown in Table 2.

### Comparative Example

The catalyst used in the Comparative Example is stannous isooctanoate, and the amount and the method for producing lactide are the same as the Example.

TABLE 1

Components of crude lactide

| | | |
|---|---|---|
| components of crude lactide (weight %) | Example | Comparative Example |
| L-lactide | 94.7 | 87.8 |
| meso-lactide | 3.1 | 10.0 |
| lactic acid | 0.7 | 0.7 |
| oligomeric lactic acid | 1.0 | 1.1 |
| color of crude lactide | white | light yellow |
| yield % (L-lactide / oligomeric lactic acid) | 88.26 | 80.96 |
| color of residual liquid | white turbid | brown turbid |

TABLE 2

Components of crude lactide after a month of circulating operation

| | | |
|---|---|---|
| components of crude lactide (weight %) | Example | Comparative Example |
| L-lactide | 91.0 | 80.2 |
| meso-lactide | 5.9 | 15.4 |
| lactic acid | 0.9 | 1.2 |
| oligomeric lactic acid | 1.4 | 1.5 |
| color of crude lactide | light yellow | dark yellow |
| yield % (L-lactide / oligomeric lactic acid) | 83.72 | 73.78 |
| color of residual liquid | dark white turbid | brown turbid |

It can be seen from Table 1 that after using the stannous salt complex catalyst, the purity of L-lactide is significantly improved, while the amount of meso-lactide is significantly reduced, and the color of the crude lactide and the residual liquid is good. It can be seen from Table 2 that the purity of L-lactide in the Example is reduced to a certain extent after all the residual liquid is recycled for one month, but the purity of L-lactide in the Comparative Example is reduced more obviously, the color of crude lactide is darker, and the color of the residual liquid turns brown. It can be seen that the stannous salt complex catalyst provided by the present disclosure can increase the purity of L-lactide, reduce the amount of meso-lactide produced, and improve the color of lactide and residual liquid.

The description of the above embodiments is only used to help understand the method and core idea of the present disclosure. It should be pointed out that for the persons skilled in the art, without departing from the principle of the present disclosure, several improvements and modifications can be made to the present disclosure, and these improvements and modifications also fall within the protection scope of the claims of the present disclosure. Various modifications to these embodiments are obvious to the persons skilled in the art, and the general principles defined herein can be implemented in other embodiments without departing from the spirit or scope of the present disclosure. Therefore, the present disclosure will not be limited to the embodiments shown in this application, but should conform to the widest scope consistent with the principles and novel features disclosed in this application.

What is claimed is:

1. A stannous salt complex catalyst, wherein comprising the following raw materials:
(1) A stannous salt
(2) A phosphite
(3) A solvent
Wherein, the mass ratio of stannous salt to phosphite is 5: 1-20:1, the mass ratio of stannous salt to solvent is 2: 1-1: 5, and the preparation steps of the stannous salt complex catalyst are as follows:
(1) Dissolving the stannous salt in the solvent and heating reflux for 30 min to fully dissolve the stannous salt to obtain a stannous salt solution;
(2) Under the protection of nitrogen, adding the phosphite dropwise to the stannous salt solution, and performing a reflux reaction for 2 h;
(3) After the reaction, extracting the solvent from the system and recycling, and obtaining a final light yellow viscous liquid which is the stannous salt complex catalyst.

2. The stannous salt complex catalyst according to claim 1, wherein the stannous salt is one or more selected from the group consisting of stannous isooctanoate, stannous chloride, stannous sulfate, stannous oxalate and stannous phosphate.

3. The stannous salt complex catalyst according to claim 1, wherein the phosphite is one or more selected from the group consisting of triphenyl phosphite, tridecyl phosphite, diphenyl phosphite, and diisodecyl pentaerythritol diphosphite.

4. The stannous salt complex catalyst according to claim 1, wherein the solvent is one or two or more of benzene, toluene, ethyl acetate, acetone and xylene.

5. The stannous salt complex catalyst according to claim 1, wherein the mass ratio of stannous salt to phosphite is 5: 1, and the mass ratio of stannous salt to solvent is 2:1.

6. A method for preparing the stannous salt complex catalyst according to claim 1, wherein comprising the following steps:
(1) Dissolving the stannous salt in the solvent and heating reflux for 30 min to fully dissolve the stannous salt to obtain a stannous salt solution;
(2) Under the protection of nitrogen, adding the phosphite dropwise to the stannous salt solution, and performing a reflux reaction for 2 h;
(3) After the reaction, extracting the solvent from the system and recycling, and obtaining a final light yellow viscous liquid which is the stannous salt complex catalyst.

7. A method for producing L-lactide using the stannous salt complex catalyst according to claim 1, wherein comprising the following steps:
(1) Continuously adding a L-lactic acid aqueous solution with a mass concentration of 80-98% to a first reactor with a rectification device, removing the free water and bound water at a temperature of 80-120° C. and a vacuum of -0.05--0.09 MPa, separating the water and lactic acid vapor produced in the reaction by a rectification tower, introducing the water vapor at the top of the tower into a collection tank after being condensed, controlling the degree of polymerization of oligomeric lactic acid to 2-8, and continuously transporting the oligomeric lactic acid to a second reactor through a pump;
(2) Equipping the second reactor with a rectification device, subjecting the oligomeric lactic acid to a further polymerization at a temperature of 120-170° C. and a vacuum degree of -0.1 MPa, controlling the degree of polymerization of the oligomeric lactic acid to 8-25, continuously transporting the oligomeric lactic acid and the continuously injected stannous salt complex catalyst to a static mixer through a pump and mixing uniformly, and then transporting a resulting mixture to a third reactor;
(3) Subjecting the oligomeric lactic acid to a cracking reaction in the third reactor with a reaction temperature of 200-230° C. and a pressure of 50-500 Pa, condensing and collecting a crude lactide vapor generated by the cracking of oligomeric lactic acid in a crude lactide storage tank, introducing an unevaporated residual liquid into a residual liquid collection tank to purify the crude lactide to obtain L-lactide with a purity required for melt polymerization.

8. The method for producing L-lactide using a stannous salt complex catalyst according to claim 7, wherein the addition amount of the stannous salt complex catalyst is 0.5-5% of the mass of the oligomeric lactic acid with a degree of polymerization of 8-25.

9. The method for producing L-lactide using a stannous salt complex catalyst according to claim 7, wherein the third reactor is a cage evaporation reactor, a scraper type film evaporator, a falling film evaporator or a rising film evaporator.

10. The method for producing L-lactide using a stannous salt complex catalyst according to claim 7, wherein the crude lactide is purified by one or several methods of melt crystallization, rectification and solvent recrystallization.

11. The method for preparing the stannous salt complex catalyst according to claim 6, wherein the stannous salt is one or more selected from the group consisting of stannous isooctanoate, stannous chloride, stannous sulfate, stannous oxalate and stannous phosphate.

12. The method for preparing the stannous salt complex catalyst according to claim 6, wherein the phosphite is one or more selected from the group consisting of triphenyl phosphite, tridecyl phosphite, diphenyl phosphite, and diisodecyl pentaerythritol diphosphite.

13. The method for preparing the stannous salt complex catalyst according to claim 6, wherein the solvent is one or two or more of benzene, toluene, ethyl acetate, acetone and xylene.

14. The method for preparing the stannous salt complex catalyst according to claim 6, wherein the mass ratio of stannous salt to phosphite is 5:1, and the mass ratio of stannous salt to solvent is 2:1.

15. The method for producing L-lactide using the stannous salt complex catalyst according to claim 7, wherein the stannous salt is one or more selected from the group consisting of stannous isooctanoate, stannous chloride, stannous sulfate, stannous oxalate and stannous phosphate.

16. The method for producing L-lactide using the stannous salt complex catalyst according to claim 7, wherein the phosphite is one or more selected from the group consisting of triphenyl phosphite, tridecyl phosphite, diphenyl phosphite, and diisodecyl pentaerythritol diphosphite.

17. The method for producing L-lactide using the stannous salt complex catalyst according to claim 7, wherein the solvent is one or two or more of benzene, toluene, ethyl acetate, acetone and xylene.

18. The method for producing L-lactide using the stannous salt complex catalyst according to claim 7, wherein the mass ratio of stannous salt to phosphite is 5:1, and the mass ratio of stannous salt to solvent is 2:1.

19. The method for producing L-lactide using a stannous salt complex catalyst according to claim 15, wherein the addition amount of the stannous salt complex catalyst is 0.5-5% of the mass of the oligomeric lactic acid with a degree of polymerization of 8-25.

20. The method for producing L-lactide using a stannous salt complex catalyst according to claim 16, wherein the addition amount of the stannous salt complex catalyst is 0.5-5% of the mass of the oligomeric lactic acid with a degree of polymerization of 8-25.
